(19) United States
(12) Patent Application Publication
(10) Pub. No.: US 2011/0263969 A1
(43) Pub. Date: Oct. 27, 2011

(54) SAR ESTIMATION IN NUCLEAR MAGNETIC RESONANCE EXAMINATION USING MICROWAVE THERMOMETRY

(76) Inventor: Jörg Ulrich Fontius, Neunkirchen A. Brand (DE)

(21) Appl. No.: 13/092,794

(22) Filed: Apr. 22, 2011

(30) Foreign Application Priority Data
Apr. 23, 2010 (DE) 10 2010 018 001.7

(51) Int. Cl. A61B 5/055

(52) U.S. Cl. 600/412

(57) ABSTRACT

The present embodiments relate to methods and devices for measuring a spatial temperature and/or SAR distribution in an examination subject in a magnetic resonance tomography device. Microwave thermosensors are provided for measuring the temperature with the aid of microwaves.

This application claims the benefit of DE 10 2010 018 001.7, filed on Apr. 23, 2010.

### BACKGROUND

The present embodiments relate to methods and devices for determining the heating of an examination subject in a magnetic resonance tomography device.

Magnetic resonance tomography devices are described, for example, in German patent application DE 102008023467.

In nuclear magnetic resonance examinations, an examination subject is heated as a result of being irradiated with radio waves (e.g., 40 MHz to 500 MHz). This increase in temperature is monitored so that no damage to tissue of the examination subject occurs. In TX array systems (e.g., systems having a plurality of RF transmit antennas), regions exhibiting an increased specific absorption rate (SAR) (e.g., hotspots) may occur in the examination subject. The hotspots are also referred to as local SAR. In contrast, global SAR may be the total radio-frequency (RF) power absorbed relative to an irradiated body mass. The local SAR may be significantly greater than the global SAR.

The SAR may be estimated by way of the global RF power absorption. This is achieved, for example, using finite element method (FEM) simulations of the electromagnetic fields in the tissue with the aid of suitable voxel models of electromagnetic parameters of the examination subject. This enables RF power limit values to be determined. These global limit values may be monitored by RF power detectors.

### SUMMARY AND DESCRIPTION

The present embodiments may obviate one or more of the drawbacks or limitations in the art. For example, SAR monitoring may be optimized in an imaging MRT system.

A microwave measurement (using microwave thermosensors measures a temperature of an examination subject with the aid of microwaves).

### BRIEF DESCRIPTION OF THE DRAWINGS

FIG. 1 shows a longitudinal section of one embodiment of an arrangement for SAR measurement using microwave thermometry;

FIG. 2 shows a cross-sectional view of one embodiment of an arrangement for SAR measurement using microwave thermometry;

FIG. 3 shows a schematic representation of the time characteristic of a thermal excitation function using RF pulses and a thermal response function of an examination subject for SAR determination by a microwave thermometry measurement; and

FIG. 4 shows a schematic overview of components of an MRT system.

### DETAILED DESCRIPTION OF THE DRAWINGS

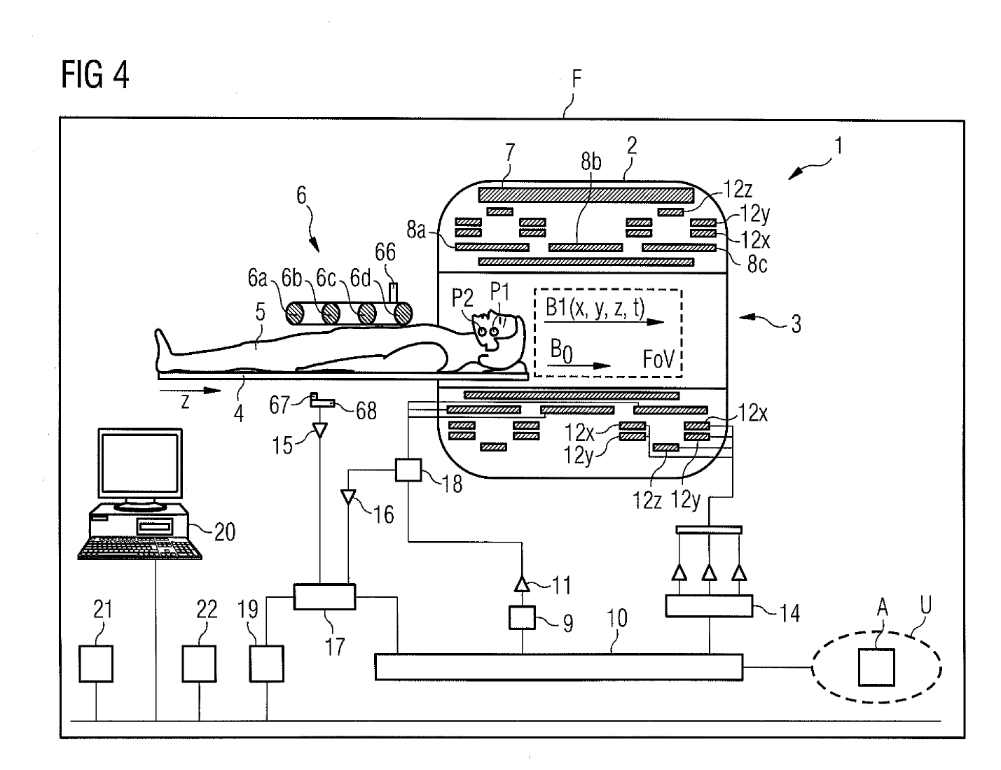

FIG. 4 shows a magnetic resonance device MRT 1 disposed in a Faraday cage F (e.g., an insulated room) and having a whole-body magnetic coil 2 with a tubular space 3, for example, in which a patient couch 4 (e.g., a patient bed) supporting an examination subject 5 (e.g., a phantom measuring element or a body) and a local coil arrangement 6 may be moved in the direction of the arrow z in order to generate images of the examination subject 5. The local coil arrangement 6 is placed on the examination subject 5. In the embodiment shown in FIG. 4, the local coil arrangement 6 (e.g., including an antenna 66 and a plurality of local coils 6a, 6b, 6c, 6d) may be used to record images in a local region (e.g., a field of view). Signals of the local coil arrangement 6 may be evaluated (e.g., converted into images and/or stored and/or displayed) by an evaluation device (e.g., elements 19, 67, 66, 15, 17) of the MRT 1. The evaluation device may be connected to the local coil arrangement 6 via coaxial cable or wirelessly.

In order to perform magnetic resonance imaging on the examination subject 5 using the magnetic resonance device MRT 1, different magnetic fields that are precisely coordinated with one another in terms of temporal and spatial characteristics, are radiated onto the examination subject.

In one embodiment, a strong magnet such as, for example, a cryomagnet 7 in a measurement chamber having the tunnel-shaped opening 3, generates a strong static main magnet field B₀ ranging from, for example, 0.2 Tesla to 3 Tesla or more. The examination subject 5 supported on the patient couch 4 is moved into a region of the main magnetic field of the magnet 7, the main magnetic field being approximately homogeneous in the area of observation or the field of view.

The magnetic resonance device MRT 1 includes gradient coils 12x, 12y, 12z, from which magnetic gradient fields B₁ (x, y, z, t) are radiated in the course of an MRT measurement of the examination subject in order to produce selective layer excitation and for spatial encoding of the measurement signal. The gradient coils 12x, 12y, 12z are controlled by a gradient coil control unit 14 that, like a pulse generation unit 9, is connected to a pulse sequence control unit 10.

The nuclear spins of atomic nuclei of the examination subject 5 are excited via magnetic radio-frequency excitation pulses B1 (x, y, z, t) that are emitted via at least one radio-frequency antenna. The at least one radio-frequency antenna is shown in FIG. 4 in simplified form as a body coil 8 including body coil segments 8a, 8b, 8c. Radio-frequency excitation pulses of the body coil segments 8a, 8b, 8c are generated by the pulse generation unit 9, which is controlled by the pulse sequence control unit 10. Following amplification by a radio-frequency amplifier 11, the radio-frequency excitation pulses are routed to the body coil 8. The radio-frequency system shown in FIG. 4 is indicated only schematically. In other embodiments, more than one pulse generation unit 9, more than one radio-frequency amplifier 11, and a plurality of radio-frequency antennas or one multipart (shown in FIG. 4 in simplified form) radio-frequency antenna (e.g., in the form of a birdcage) having different numbers of radio-frequency antenna elements 8a, 8b, 8c are used in the magnetic resonance device MRT 1.

The radio-frequency antenna shown as the body coil 8 in FIG. 4 may include a plurality of transmit channels 8a, 8b, 8c, each transmit channel of the plurality of transmit channels emitting radio-frequency excitation pulses.

Fractions of the total field B1 (x,y,z,t) or the non-stationary (without B0) total field may also be emitted in the form of radio-frequency excitation pulses by the transmit channels 6a, 6b, 6c, 6d of the local coil 6. Fractions of the non-stationary total field B1 (x,y,z,t) may also be generated in the form of gradient fields by the gradient coil channels 12x, 12y, 12z.

Signals transmitted by the excited nuclear spins are received by the body coil 8 and/or by the local coils 6a, 6b, 6c, 6d, amplified by associated radio-frequency preamplifiers 15, 16, and processed further and digitized by a receiving unit 17. The recorded measured data is digitized and stored in the form of complex numeric values in a k-space matrix. An associated MR image may be reconstructed from the k-space matrix populated with values using a multidimensional Fourier transform.

In the case of a coil that may be operated both in transmit and in receive mode (e.g., the body coil 8), correct signal forwarding is controlled by an upstream duplexer 18.

An image processing unit 19 generates an image from the measured data. The image is displayed to a user via an operator console 20 and/or stored in a memory unit 21. A central computer unit 22 controls the individual system components.

The present embodiments is not used for diagnosis of a body, per se. Rather, using microwave thermometry and an evaluation, the location of hotspots that occur in the case of specific RF pulses and/or how great specific absorption rate (SAR) absorption is in absolute terms or relative terms to the surroundings or the body, may be determined on a dummy, a human body or an animal.

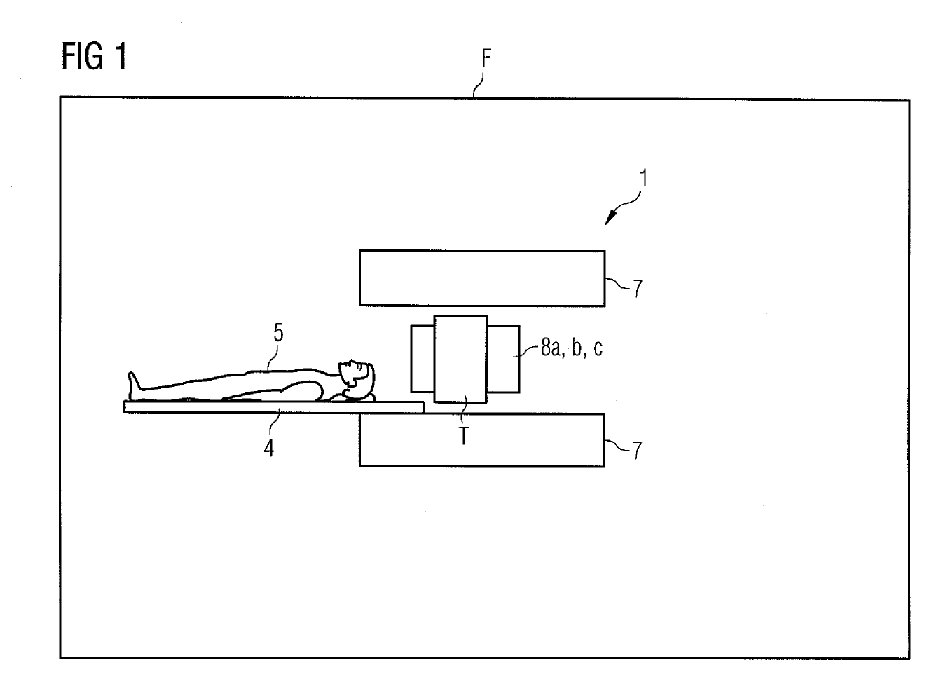

FIG. 1 shows a longitudinal section of one embodiment of an arrangement for SAR measurement on the examination subject 5 in the MRT 1 using microwave thermometry thermosensors T.

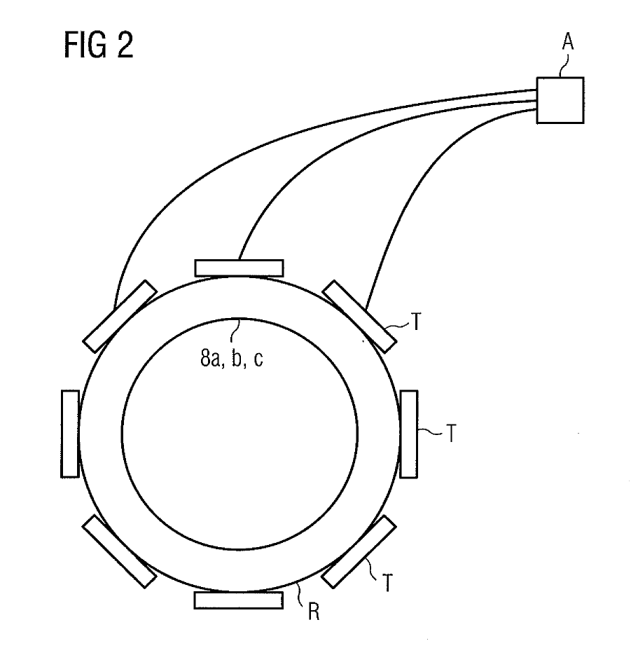

FIG. 2 shows a cross-sectional view of one embodiment of an arrangement of the microwave thermometry thermosensors T, the microwave thermometry thermosensors T being disposed on an annular carrier arrangement R (e.g., between, inside or outside of the coils 8a, 8b, 8c).

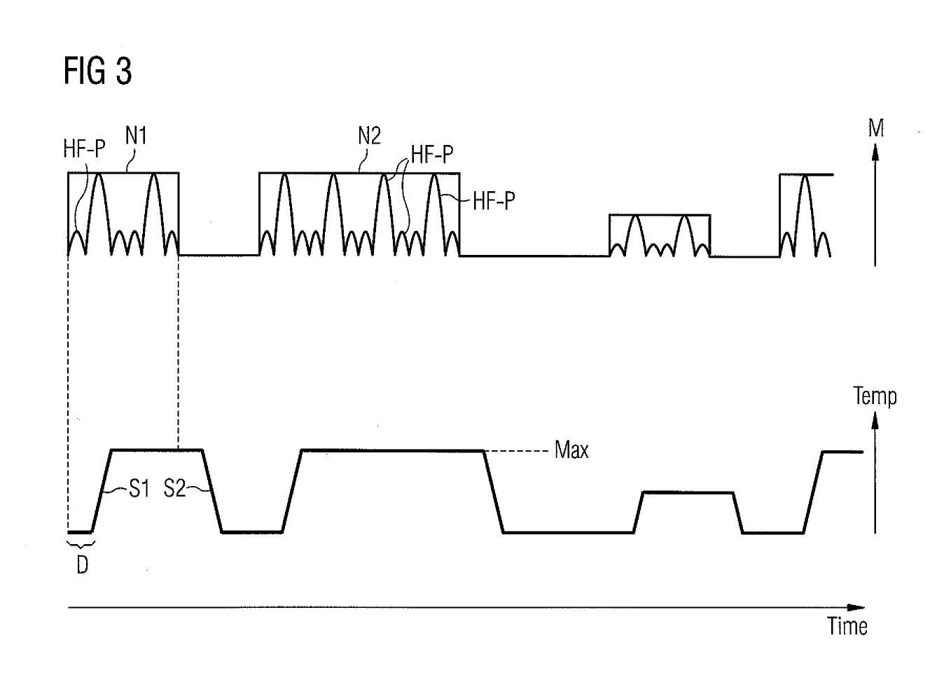

In a top section, FIG. 3 shows a schematic representation of the time characteristic of a thermal excitation function M consisting of RF pulses HF-P that act on the examination subject 5 in the MRT 1; in a bottom section, FIG. 3 shows a thermal response function Temp (e.g., thermal radiation of the examination subject 5 to a plurality of thermosensors) measured (using microwave thermometry) using one or more of the microwave thermometry thermosensors T. For the SAR measurement, the response functions measured by the microwave thermometry thermosensors T are analyzed in order to determine a temperature profile at one or more points in the examination subject and/or to detect hotspots (e.g., points in the examination subject that are hotter than the surroundings) in the examination subject 5.

A depicted temperature profile Temp of the examination subject 5 is delayed in time by a time D with respect to the RF pulses HF-P triggering a rise in temperature. The temperature profile Temp determined by at least one of the microwave thermometry thermosensors T may reveal more a response to an (assumed) envelope curve M of the RF pulses HF-P than to each individual RF pulse HF-P in terms of a resolution.

The temperature profile Temp shows a rise S1 (slope) that occurs (delayed by D) after the start of a pulse sequence N1 and shows a fall S2 that occurs (delayed by D) after the end of the pulse sequence N1.

The method described below uses non-invasive measurements of the temperature of the examination subject during an (prescan and/or imaging) MR measurement using microwave thermometry.

Microwave thermometry has the advantage that temperatures may also be measured non-invasively in deeper-lying areas of the examination subject; see, without actual reference to MRT imaging, articles such as, for example:
Hand, J. W., et al., “Monitoring of deep brain temperature in infants using multi-frequency microwave radiometry and thermal modeling,” Physics in Medicine and Biology, Vol. 46, No. 7, 2001;
Bri, S., et al., “Experimental evaluation of new thermal inversion approach in correlation microwave thermometry [tumor detection],” Electronics Letters, Vol. 36, No. 5, 2000: pp. 439-440;
Bruggmoser, G., et al., “Experimental hyperthermia of nude mice controlled by microwave thermometry,” European Surgery, Vol. 24, No. 4, 1992: pp. 199-200;
N. M. Nedeltchev, “Thermal microwave emission depth and soil moisture remote sensing,” International Journal of Remote Sensing, Vol. 20, No. 11, 1999: pp: 2183-2194; and
“Guide to Microwave Temperature Measurement,” Loma Systems, Apr. 21, 2011: http://www.loma.com/lo_tempmeas_guide.shtml.

Hotspots potentially occurring in the course of an MRT examination may be located in deeper-lying regions of the examined examination subject and may be detected by a microwave thermometry measurement. A measurement setup according to FIGS. 1 and 2 is proposed as an exemplary embodiment.

An array setup (e.g., an arrangement of a plurality of microwave thermosensors T) is used, for example. Tomographic evaluation methods such as, for example, projection methods may be used to increase the spatial resolution of the thermal distribution as well as the sensitivity.

In one embodiment, the thermosensors T according to FIG. 2 are arranged such that the thermosensors T enclose a measurement volume (e.g., the FoV) in the manner of, for example, an annular arrangement (e.g., a ring inside or outside of RF coils 8a-c of the MRT) on an annular carrier R in an MRT.

In order to minimize external sources of interference, the RF cage F (as shown in FIG. 4) of an MR chamber is configured such that the RF cage F also shields against sources of microwave interference. Microwave shields U may also be installed in addition to or instead of the RF cage F on the MR system 1 (e.g., for electronic modules shielded using shields).

The heating of the examination subject 5 takes place at an RF energy that is radiated, for example, by the MR transmit coils 8a-c used in an MR examination (e.g., a microwave thermometry prescan examination preceding the measurement) and is absorbed in the examination subject 5.

A prescan MR examination (at least also) including measurement of the temperature rise occurring as a result of microwave thermometry may apply the RF pulse shapes planned for one or more succeeding imaging acquisitions. This causes local hotspots to form in the examined body 5. The hotspots are coil- and RF-pulse-specific and may be detected by the thermosensors T.

The measurement method uses, for example, lock-in technology, the basis of which is that the signal to be measured, defined by a physical effect, is modulated in time and demodulated with a cross-correlation so that the physical effect is filtered out, and interference signals (noise) are suppressed. In this way, the signal noise may be amplified by orders of magnitude, and the measurement becomes very sensitive.

In the present method, the temperature distribution in the examination subject may be modulated in time by emitting the RF pulses in a first MR examination in packets of different length, pauses and amplitudes. In one embodiment, the pattern is a pseudo-random sequence that may be suitable for cross-correlation (see FIG. 3).

In addition to the cross-correlation, a transmission function that takes into account a delay in the temperature rise or temperature fall (delay D) and/or a rising and/or falling edge (slope S1, S2) may also be included. As FIG. 3 shows, the same or similar RF pulses HF-P planned as pulse sequences of a subsequent MRT imaging acquisition are packed into a modulation pulse N of a modulation curve M.

Based on an array arrangement of the sensors, a 2D/3D image of the temperature distribution, for example, may be computed in an evaluation device (e.g., a computer) A using, for example, projection reconstruction, and a position of hotspots P1 in the examination subject 5 may be identified. A “local SAR to global SAW” factor may be determined by comparison of the hotspot SAR intensity relative to the background.

The global SAR may be determined relatively accurately through measurement of the globally absorbed RF power in accordance with conventional methods. The local SAR may be estimated based on the determined factor, local SAR to global SAR.

The SAR estimation may be performed as an “SAR adjustment” (in a prescan MRT measurement) prior to each imaging MRT measurement or also online during the imaging MRT measurement.

Possible advantages are:
Patient-specific SAR estimation;
More accurate SAR estimation, lower error tolerances;
Coil-specific SAR estimation;
Pulse-sequence-specific SAR estimation;
Passive (without transmission of microwaves), non-invasive method; and
A microwave frequency measurement permits measurement of deeper-lying regions.

Possible examples of a (microwave) thermosensor (although the most diverse other types that the person skilled in the art finds) include the products of the company Loma. For example, products for monitoring the temperature of foodstuffs (see e.g., http://www.loma.co.uk/lo_{—} temperature_measurement.shtml) may be used as microwave thermosensors.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for determining a heating of an examination subject in a magnetic resonance tomography (MRT) device, the method comprising:
transmitting, with the MRT device, radio-frequency (RF) pulses; and
determining the heating of the examination subject using a plurality of thermosensors.

2. The method as claimed in claim 1, wherein the plurality of thermosensors is configured for measuring microwave radiation.

3. The method as claimed in claim 1, wherein the plurality of thermosensors is arranged such that the plurality of thermosensors encloses a measurement volume in the examination subject.

4. The method as claimed in claim 1, wherein microwaves emitted from regions below a surface of the examination subject are measured using the plurality of thermosensors.

5. The method as claimed in claim 1, further comprising determining a heating of a plurality of regions below a surface of the examination subject.

6. The method as claimed in claim 5, further comprising determining a maximum heating of the plurality of regions within the examination subject.

7. The method as claimed in claim 1, further comprising determining a spatial distribution of a specific absorption rate (SAR) in the examination subject taking into account temperature radiation measured by the plurality of thermosensors and taking into account energy emitted by the MRT device by the RF pulses, energy distribution, or the RF pulses and the energy distribution.

8. The method as claimed in claim 1, wherein the examination subject is heated by the RF pulses, the RF pulses being emitted by at least one magnetic resonance transmit coil.

9. The method as claimed in claim 7, wherein, prior to an imaging MRT acquisition of the examination subject, shapes of RF pulses that are planned for a subsequent imaging MRT acquisition are applied for measuring the spatial SAR distribution in the examination subject.

10. The method as claimed in claim 5, further comprising:
performing a microwave thermometry measurement using the plurality of thermosensors during an imaging MRT acquisition of the examination subject; and
determining the heating of the plurality of regions in the examination subject.

11. The method as claimed in claim 1, further comprising:
performing microwave thermometry measurements on the examination subject using different coils, the RF pulses, or the different coils and the RF pulses; and
storing results produced from the performed microwave thermometry measurements,
wherein the results are taken into account for determining an anticipated heating of regions, for specifying a pulse amplitude in a subsequent imaging acquisition of the examination subject, or for determining the anticipated heating of the regions and specifying the pulse amplitude in the subsequent imaging acquisition of the examination subject, the determining the anticipated heating, the specifying, or the determining the anticipated heating and the specifying being a function of coils, the RF pulses, or the coils and the RF pulses.

12. The method as claimed in claim 1, wherein a temperature distribution in the examination subject is modulated in time by emitting the RF pulses in packets of different length, pauses, or amplitudes.

13. The method as claimed in claim 12, wherein a pattern of the emitted RF pulses is a pseudo-random sequence that is used for a cross-correlation.

14. The method as claimed in claim 1, wherein in order to determine a spatial specific absorption rate (SAR) distribution in the examination subject, one or more of a delay in a temperature rise, a delay in a temperature fall, a shape of a rising edge, and a shape of a falling edge is taken into account.

15. The method as claimed in claim 1, further comprising:
computing a spatial temperature distribution in the examination subject using a projection reconstruction; and
identifying positions of hotspots in the examination subject.

16. The method as claimed in claim 1, further comprising determining a ratio of a local specific absorption rate (SAR) at a hotspot to a global SAR in the examination subject by comparison of a hotspot intensity relative to a background.

17. The method as claimed in claim 16, wherein the global SAR in the examination subject is determined through measurement of an RF power absorbed in the whole examination subject.

18. The method as claimed in claim 1, wherein at least one maximum of a specific absorption rate in the examination subject is determined and taken into account for specifying pulses in a subsequent imaging acquisition of the examination subject.

19. A device for determining the heating in an examination subject induced by a plurality of radio-frequency (RF) pulses of a magnetic resonance tomography (MRT) device, the device comprising:
thermosensors.

20. The device as claimed in claim 19, wherein the thermosensors comprise a plurality of microwave thermosensors.

21. The device as claimed in claim 19, wherein the thermosensors are arranged such that the thermosensors enclose a measurement volume in the MRT device.

22. The device as claimed in claim 19, further comprising an RF cage of the MRT device, the RF cage configured to shield again microwaves from outside of the RF cage.

23. The device as claimed in claim 19, further comprising microwave shields installed in the MRT device as shields on electronic modules of the MRT device.

24. The device as claimed in claim 19, wherein the device is configured such that, prior to an imaging acquisition of the examination subject, shapes of RF pulses planned for a subsequent imaging acquisition are also applied by a device for determining a spatial temperature distribution, a specific absorption rate (SAR) distribution in the examination subject, or the spatial temperature distribution and the SAR distribution in the examination subject.

25. The device as claimed in claim 20, wherein the plurality of microwave thermosensors is configured to measure microwaves emitted from positions below a surface of the examination subject.

26. The device as claimed in claim 19, further comprising a computer, the computer configured for determining the heating of a plurality of regions of the examination subject.

27. The device as claimed in claim 19, further comprising a computer, the computer configured for determining a specific absorption rate (SAR) in a plurality of regions inside the examination subject.

28. The device as claimed in claim 27, wherein the computer is configured for determining a spatial distribution of the SAR in the examination subject taking into account temperature radiation measured by microwave thermosensors and taking into account energy emitted by the MRT device by the plurality of RF pulses, an energy distribution, or the plurality of RF pulses and the energy distribution.

29. The device as claimed in claim 20, further comprising a computer, the computer configured for microwave thermometry measurement using the plurality of microwave thermosensors and being configured for determining the heating of the examination subject during an imaging MRT acquisition of the examination subject.

30. The device as claimed in claim 19, further comprising a computer, the computer configured for taking into account results of microwave thermometry measurements prior to an imaging acquisition to specify shapes, amplitudes, or the shapes and the amplitudes of the plurality of RF pulses during the imaging acquisition of the examination subject.

31. The device as claimed in claim 19, further comprising a modulating device, the modulating device configured to modulate a temperature distribution in the examination subject in time by emitting the plurality of RF pulses in packets of different length, pauses or amplitudes.

32. The device as claimed in claim 30, wherein the computer is configured for taking into account a delay in a temperature rise, a temperature fall, a rising edge, or falling edge of the heating to determine a spatial specific absorption rate (SAR) distribution in the examination subject.

33. The device as claimed in claim 19, further comprising a computer, the computer configured to:
compute a spatial temperature distribution in the examination subject using a projection reconstruction; and
identify positions of hotspots in the examination subject.

34. The device as claimed in claim 19, further comprising a computer, the computer configured for determining a ratio of local specific absorption rate (SAR) at a hotspot position to a global SAR in the examination subject by comparison of measured temperature data at the hotspot position relative to the environment.

35. The device as claimed in claim 19, further comprising a computer, the computer configured for determining at least one maximum of a specific absorption rate (SAR) in the examination subject and configured for taking the at least one maximum of the SAR into account to specify shapes, amplitudes of the plurality of pulses, or the shapes and the amplitudes of the plurality of pulses in a subsequent imaging acquisition of the examination subject.

36. The device as claimed in claim 21, wherein the thermosensors are arranged such that the thermosensors enclose a measurement volume in the MRT device in an annular arrangement.
